SEQUENCE LISTING (43 sequences)

SEQ ID NO: 1 - protein, 267 aa, synthetic construct
  REGION 1..267: note = Human KAI1 protein
MGSACIKVTKYFLFLFNLIFFILGAVILGFGVWILADKSSFISVLQTSSSSLRMGAYVFIGVGAVTMLMGFLGCIGAVNEVRCLLGLYFAFLLLILIAQVTAGALFYFNMGKLKQEMGGIVTELIRDYNSSREDSLQDAWDYVQAQVKCCGWVSFYNWTDNAELMNRPEVTYPCSCEVKGEEDNSLSVRKGFCEAPGNRTQSGNHPEDWPVYQEGCMEKVQAWLQENLGIILGVGVGVAIIELLGMVLSICLCRHVHSEDYSKVPKY

SEQ ID NO: 2 - protein, 20 aa, synthetic construct
  REGION 1..20: note = Wild type KAI1 polypeptide
NRPEVTYPCSCEVKGEEDNS

SEQ ID NO: 3 - protein, 20 aa, synthetic construct
  REGION 1..20: note = Mutant KAI1 polypeptide
NRPEVTAPASCEVKGAAANS

SEQ ID NO: 4 - protein, 118 aa, synthetic construct
  REGION 1..118: note = Extracellular domain of recombinant human KAI1 protein
GKLKQEMGGIVTELIRDYNSSREDSLQDAWDYVQAQVKCCGWVSFYNWTDNAELMNRPEVTYPCSCEVKGEEDNSLSVRKGFCEAPGNRTQSGNHPEDWPVYQEGCMEKVQAWLQENL

SEQ ID NO: 5 - DNA, 22 bp, synthetic construct
  misc_feature 1..22: note = Forward primer for amplifying TGF-beta1
  source 1..22: mol_type = other DNA
tgatacgcctgagtggctgtct

SEQ ID NO: 6 - DNA, 22 bp, synthetic construct
  misc_feature 1..22: note = Reverse primer for amplifying TGF-beta1
  source 1..22: mol_type = other DNA
cacaagagcagtgagcgctgaa

SEQ ID NO: 7 - DNA, 23 bp, synthetic construct
  misc_feature 1..23: note = Forward primer for amplifying fibronectin
  source 1..23: mol_type = other DNA
ccctatctctgataccgttgtcc

SEQ ID NO: 8 - DNA, 22 bp, synthetic construct
  misc_feature 1..22: note = Reverse primer for amplifying fibronectin
  source 1..22: mol_type = other DNA
tgccgcaactactgtgattcgg

SEQ ID NO: 9 - DNA, 22 bp, synthetic construct
  misc_feature 1..22: note = Forward primer for amplifying Col1a1
  source 1..22: mol_type = other DNA
cctcagggtattgctggacaac

SEQ ID NO: 10 - DNA, 22 bp, synthetic construct
  misc_feature 1..22: note = Reverse primer for amplifying Col1a1
  source 1..22: mol_type = other DNA
cagaaggaccttgtttgccagg

SEQ ID NO: 11 - DNA, 23 bp, synthetic construct
  misc_feature 1..23: note = Forward primer for amplifying GAPDH
  source 1..23: mol_type = other DNA
catcactgccacccagaagactg

SEQ ID NO: 12 - DNA, 23 bp, synthetic construct
  misc_feature 1..23: note = Reverse primer for amplifying GAPDH
  source 1..23: mol_type = other DNA
atgccagtgagcttcccgttcag

SEQ ID NO: 13 - DNA, 22 bp, synthetic construct
  misc_feature 1..22: note = Forward primer for amplifying alpha-SMA
  source 1..22: mol_type = other DNA
tgctgacagaggcaccactgaa

SEQ ID NO: 14 - DNA, 23 bp, synthetic construct
  misc_feature 1..23: note = Reverse primer for amplifying alpha-SMA
  source 1..23: mol_type = other DNA
cagttgtacgtccagaggcatag

SEQ ID NO: 15 - DNA, 24 bp, synthetic construct
  misc_feature 1..24: note = Forward primer for amplifying TIMP1
  source 1..24: mol_type = other DNA
gatatgcccacaagtcccagaacc

SEQ ID NO: 16 - DNA, 23 bp, synthetic construct
  misc_feature 1..23: note = Reverse primer for amplifying TIMP1
  source 1..23: mol_type = other DNA
gcacaccccacagccagcactat

SEQ ID NO: 17 - DNA, 22 bp, synthetic construct
  misc_feature 1..22: note = Forward primer for amplifying MMP9
  source 1..22: mol_type = other DNA
gctgactacgataaggacggca

SEQ ID NO: 18 - DNA, 22 bp, synthetic construct
  misc_feature 1..22: note = Reverse primer for amplifying MMP9
  source 1..22: mol_type = other DNA
tagtggtgcaggcagagtagga

SEQ ID NO: 19 - DNA, 22 bp, synthetic construct
  misc_feature 1..22: note = Forward primer for amplifying Vimentin
  source 1..22: mol_type = other DNA
cggaaagtggaatccttgcagg

SEQ ID NO: 20 - DNA, 22 bp, synthetic construct
  misc_feature 1..22: note = Reverse primer for amplifying Vimentin
  source 1..22: mol_type = other DNA
agcagtgaggtcaggcttggaa

SEQ ID NO: 21 - DNA, 20 bp, synthetic construct
  misc_feature 1..20: note = Forward primer for amplifying human vimentin
  source 1..20: mol_type = other DNA
ttgtggcagggttgatgtta

SEQ ID NO: 22 - DNA, 20 bp, synthetic construct
  misc_feature 1..20: note = Reverse primer for amplifying human vimentin
  source 1..20: mol_type = other DNA
cacccactccaactccaact

SEQ ID NO: 23 - DNA, 23 bp, synthetic construct
  misc_feature 1..23: note = Forward primer for amplifying human Col1a1
  source 1..23: mol_type = other DNA
ttatagagcgatacaagggggag

SEQ ID NO: 24 - DNA, 23 bp, synthetic construct
  misc_feature 1..23: note = Reverse primer for amplifying human Col1a1
  source 1..23: mol_type = other DNA
cgccgtctgattatcttgatgag

SEQ ID NO: 25 - DNA, 21 bp, synthetic construct
  misc_feature 1..21: note = Forward primer for amplifying human TGF-beta
  source 1..21: mol_type = other DNA
gaggctaccctagacacaagg

SEQ ID NO: 26 - DNA, 20 bp, synthetic construct
  misc_feature 1..20: note = Reverse primer for amplifying human TGF-beta
  source 1..20: mol_type = other DNA
gggtgccgttgctcatcata

SEQ ID NO: 27 - DNA, 20 bp, synthetic construct
  misc_feature 1..20: note = Forward primer for amplifying human alpha-SMA
  source 1..20: mol_type = other DNA
tgaactccaacgtcaagcgg

SEQ ID NO: 28 - DNA, 23 bp, synthetic construct
  misc_feature 1..23: note = Reverse primer for amplifying human alpha-SMA
  source 1..23: mol_type = other DNA
cgccgtctgattatcttgatgag

SEQ ID NO: 29 - DNA, 20 bp, synthetic construct
  misc_feature 1..20: note = Forward primer for amplifying human GFAP
  source 1..20: mol_type = other DNA
ctggaggttgagagggacaa

SEQ ID NO: 30 - DNA, 19 bp, synthetic construct
  misc_feature 1..19: note = Reverse primer for amplifying human GFAP
  source 1..19: mol_type = other DNA
cagcctcaggtggtttcat

SEQ ID NO: 31 - DNA, 22 bp, synthetic construct
  misc_feature 1..22: note = Forward primer for amplifying human 9S
  source 1..22: mol_type = other DNA
gactccggaacaaacgtgaggt

SEQ ID NO: 32 - DNA, 20 bp, synthetic construct
  misc_feature 1..20: note = Reverse primer for amplifying human 9S
  source 1..20: mol_type = other DNA
cttcatcttgccctcgtcca

SEQ ID NO: 33 - DNA, 20 bp, synthetic construct
  misc_feature 1..20: note = Forward primer for amplifying human HGMA1
  source 1..20: mol_type = other DNA
caactccaggaaggaaacca

SEQ ID NO: 34 - DNA, 18 bp, synthetic construct
  misc_feature 1..18: note = Reverse primer for amplifying human HGMA1
  source 1..18: mol_type = other DNA
aggactcctgcgagatgc

SEQ ID NO: 35 - DNA, 20 bp, synthetic construct
  misc_feature 1..20: note = Forward primer for amplifying human p16
  source 1..20: mol_type = other DNA
caccgaatagttacggtcgg

SEQ ID NO: 36 - DNA, 20 bp, synthetic construct
  misc_feature 1..20: note = Reverse primer for amplifying human p16
  source 1..20: mol_type = other DNA
gcacgggtcgggtgagagtg

SEQ ID NO: 37 - DNA, 25 bp, synthetic construct
  misc_feature 1..25: note = Forward primer for amplifying human p21
  source 1..25: mol_type = other DNA
catgtggacctgtcactgtcttgta

SEQ ID NO: 38 - DNA, 19 bp, synthetic construct
  misc_feature 1..19: note = Reverse primer for amplifying human p21
  source 1..19: mol_type = other DNA
gaagatcagccggcgtttg

SEQ ID NO: 39 - DNA, 22 bp, synthetic construct
  misc_feature 1..22: note = Forward primer for amplifying human p53
  source 1..22: mol_type = other DNA
cctcagcatcttatccgagtgg

SEQ ID NO: 40 - DNA, 21 bp, synthetic construct
  misc_feature 1..21: note = Reverse primer for amplifying human p53
  source 1..21: mol_type = other DNA
tggatggtggtacagtcaggc

SEQ ID NO: 41 - DNA, 21 bp, synthetic construct
  misc_feature 1..21: note = Forward primer for amplifying human SIRT1
  source 1..21: mol_type = other DNA
tgctggcctaatagagtggca

SEQ ID NO: 42 - DNA, 20 bp, synthetic construct
  misc_feature 1..20: note = Reverse primer for amplifying human SIRT1
  source 1..20: mol_type = other DNA
ctcagcgccatggaaaatgt

SEQ ID NO: 43 - protein, 12 aa, synthetic construct
  REGION 1..12: note = fragment of KAI1 protein
  VARIANT 2: note = X can be Ala, Asp, Glu, Gly, Phe, Leu, Ser, Tyr, Cys, Trp, Pro,His, Gln, Arg, Ile, Met, Asn, Lys, Ser or Val.
  VARIANT 4..7: note = X can be Ala, Asp, Glu, Gly, Phe, Leu, Ser, Tyr, Cys, Trp, Pro,His, Gln, Arg, Ile, Met, Asn, Lys, Ser or Val.
YXCXXXXXXEED

(19) United States
(12) Patent Application Publication
(10) Pub. No.: US 2024/0382559 A1
(43) Pub. Date: Nov. 21, 2024

(54) PHARMACEUTICAL COMPOSITION COMPRISING KAI1 POLYPEPTIDE FOR INHIBITION OF HEPATIC FIBROSIS AND USE THEREOF

(71) Applicant: PUSAN NATIONAL UNIVERSITY INDUSTRY-UNIVERSITY COOPERATION FOUNDATION, Busan (KR)

(72) Inventors: Jin HUR, Yangsan-si, Gyeongsangnam-do (KR); Jung-Hwa HAN, Yangsan-si, Gyeongsangnam-do (KR); Soo Yong LEE, Yangsan-si, Gyeongsangnam-do (KR)

(21) Appl. No.: 18/688,285

(22) PCT Filed: Aug. 31, 2022
(86) PCT No.: PCT/KR2022/013013

§ 371 Date: Feb. 29, 2024

(30) Foreign Application Priority Data
Aug. 31, 2021 (KR) 10-2021-0115891

(51) Int. Cl. A61K 38/17; A61K 38/10; A61P 1/16

(52) U.S. Cl. CPC A61K 38/17; A61K 38/10; A61P 1/16

(57) ABSTRACT

The KAI1 protein or a fragment thereof according to the present invention inhibited hepatic fibrosis in mice in which acute hepatic fibrosis was induced by CCl₄. The KAI1 protein suppressed hepatic fibrosis by inhibiting fibrosis of hepatocytes treated with TGF-β1 and promoting senescence of hepatic stellate cells. Therefore, the pharmaceutical composition comprising the KAI1 protein according to the present invention may be used to effectively prevent or treat hepatic fibrosis.

### TECHNICAL FIELD

The present invention relates to a pharmaceutical composition for inhibiting hepatic fibrosis comprising KAI1 polypeptide and a fragment thereof, and a method for preventing or treating hepatic fibrosis using the same.

### BACKGROUND ART

The liver is called a silent organ, and even if a disease develops in the liver, early detection of the disease is difficult, making early detection and treatment difficult. Liver cancer is a very dangerous cancer that ranks second in male cancer mortality. Liver cancer occurs when a significant number of people have hepatic cirrhosis (fibrosis and hardening of the liver) and develop into cancer. Hepatic fibrosis develops into hepatic cirrhosis, liver cancer, and eventually death due to liver failure.

There is still no standard treatment for hepatic fibrosis, and no therapeutic agent that has an obvious effect has been developed. Recently, clinical research is in progress based on the basic mechanisms of inflammation and fibrosis studied through preclinical studies. Specifically, drugs that inhibit hepatic stellate cell activation, such as antioxidants and interferons, drugs that inhibit TGF-β cytokines, and drugs that inhibit COX2 are being tested with the goal of reducing inflammation in the liver and regulating immune responses (Zui Tan et al., Front Cell Dev Biol, 2021, 9:730176).

Therefore, it is important to prevent the progression of chronic hepatitis to hepatic cirrhosis, and there is a need to develop a therapeutic agent that can inhibit hepatic fibrosis, a process that progresses from hepatitis to hepatic cirrhosis.

### DETAILED DESCRIPTION OF INVENTION

### Technical Problem

Accordingly, the present inventors studied a method for effectively inhibiting hepatic fibrosis and confirmed that KAI1 protein or a fragment thereof may inhibit hepatic fibrosis at the cellular and animal levels. Based on the above, the present invention was completed.

### Solution to Problem

In order to solve the above problem, in one aspect of the present invention, there is provided a pharmaceutical composition for preventing or treating hepatic fibrosis, comprising KAI1 polypeptide and a fragment thereof.

In another aspect of the present invention, there is provided a method for preventing or treating hepatic fibrosis, comprising administering to a subject the pharmaceutical composition.

In another aspect of the present invention, there is provided the use of the pharmaceutical composition for the prevention or treatment of hepatic fibrosis.

### EFFECTS OF INVENTION

The KAI1 protein or a fragment thereof according to the present invention inhibited hepatic fibrosis in mice in which acute hepatic fibrosis was induced by CCl4. The KAI1 protein or a fragment thereof suppressed hepatic fibrosis by inhibiting fibrosis of hepatocytes treated with TGF-β1 and promoting senescence of hepatic stellate cells. Therefore, a pharmaceutical composition comprising the KAI1 protein according to the present invention or a fragment thereof can be used to effectively prevent or treat hepatic fibrosis.

### BRIEF DESCRIPTION OF DRAWINGS

FIG. 1 is a schematic diagram of KAI1 peptide; and a schematic diagram showing the possible binding site between the KAI1 peptide fragment and VEGF (vascular endothelial growth factor) or PDGF (platelet-derived growth factor).

FIG. 2 is a diagram showing an experimental schedule to verify the effect of inhibiting hepatic fibrosis by KAI1 protein.

FIG. 3 is a diagram showing the results observed by H&E staining of liver tissue after treatment with KAI1 protein in acute hepatic fibrosis model mice.

FIG. 4 is a graph showing the results of confirming the gene expression of Col1a1,fibronectin, and TGFβ1 in liver tissue after treatment with KAI1 protein in acute hepatic fibrosis model mice by qPCR analysis. *, p<0.05 vs. veh, #, p<0.05; ##, p<0.01 vs. CCl₄

FIG. 5 is a graph showing the results of confirming the gene expression of α-SMA, Col1a1, TIMP1, and MMP9 according to treatment with KAI1 protein by qPCR analysis when hepatic fibrosis was induced by treating liver cell lines with TGF-β1. *, p<0.05 vs. veh, #, p<0.05; ##, p<0.01 vs. TGF-β

FIG. 6 is a diagram showing the results of confirming the protein expression of ZO-1 according to treatment with KAI1 protein by immunoblotting when hepatic fibrosis was induced by treating liver cell lines with TGF-β1.

FIG. 7 is a graph showing the results of confirming the changes in gene expression of α-SMA, Col1a1, TIMP1, Vimentin, TGF-β, and fibronectin according to treatment with recombinant human KAI1 protein (rhKAI1), wild type KAI1 peptide (pepKAI1 WT) or mutant KAI1 peptide (pepKAI1 Mut) by qPCR analysis when hepatic fibrosis was induced by treating liver cell lines with TGF-β1. *, p<0.05 vs. veh, #, p<0.05; ##, p<0.01 vs. TGFβ

FIG. 8 is a graph showing the results of confirming cell proliferation according to treatment with KAI1 protein in hepatic stellate cell lines, and the results of confirming the changes in gene expression of Vimentin, Col1a1, TGF-B1, α-SMA, and GFAP by qPCR analysis.

FIG. 9 is a diagram showing the results of confirming the protein expression of Vimentin, Col1α1, α-SMA, and GFAP according to treatment with KAI1 protein in hepatic stellate cell lines by immunoblotting.

FIG. 10 is a graph showing the results of quantifying the protein expression of Vimentin, Col1a1, α-SMA, and GFAP according to treatment with KAI1 protein in hepatic stellate cell lines. *, p<0.05; **, p<0.01 vs. veh (24 h) , #, p<0.05; ##, p<0.01 vs. veh (48 h)

FIG. 11 is a graph showing the results of confirming the changes in gene expression of HGMA1, p16, p21, p53, and SIRT1 according to treatment with KAI1 protein in hepatic stellate cell lines by qPCR analysis. *, p<0.05; **, p<0.01 vs. veh (24 h) , #, p<0.05; ##, p<0.01 vs. veh (48 h)

FIG. 12 is a diagram showing the results of confirming the protein expression of SIRT1 and p16 according to treatment with KAI1 protein in hepatic stellate cell lines by immunoblotting.

FIG. 13 is a graph showing the results of quantifying the protein expression of SIRT1 and p16 according to treatment with KAI1 protein in hepatic stellate cell lines. *, p<0.05; **, p<0.01 vs. veh (24 h), #, p<0.05; ##, p<0.01 vs. veh (48 h)

### BEST MODE FOR CARRYING OUT THE INVENTION

In one aspect of the present invention, there is provided a pharmaceutical composition for preventing or treating hepatic fibrosis, comprising KAI1 protein or a fragment thereof.

As used herein, the term “KAI1” protein is also referred to as CD82, CD82 antigen, 4F9, C33, GR15, IA4, inducible membrane protein R2, SAR2, ST6 (suppressor of tumorigenicity 6) or tetraspanin-27 (TSPAN27). The KAI1 protein may include a protein having the amino acid sequence of UniProt Accession No. P27701 (human) or P40237 (mouse). The KAI1 protein may comprise a polypeptide consisting of the amino acid sequence of SEQ ID NO: 1.

The fragment of the KAI1 protein is a part of the KAI1 protein and may be a polypeptide having the activity of the KAI1 protein. The fragment of the KAI1 protein may be the extracellular domain of the KAI1 protein. The fragment of the KAI1 protein may comprise a polypeptide consisting of the 33rd to 53rd amino acid sequence from the N terminus in a polypeptide consisting of the amino acid sequence of SEQ ID NO: 1. In addition, the fragment of the KAI1 protein may comprise a polypeptide consisting of the 101st to 228th amino acid sequence from the N terminus in a polypeptide consisting of the amino acid sequence of SEQ ID NO: 1. In addition, the fragment of the KAI1 protein may comprise a polypeptide consisting of the 111st to 228th amino acid sequence from the N terminus in a polypeptide consisting of the amino acid sequence of SEQ ID NO: 1. In addition, the fragment of the KAI1 protein may comprise a polypeptide consisting of the 166th to 185th amino acid sequence from the N terminus in a polypeptide consisting of the amino acid sequence of SEQ ID NO: 1.

Specifically, the fragment of the KAI1 protein may comprise a polypeptide consisting of the amino acid sequence of SEQ ID NO: 2 or SEQ ID NO: 4.

In addition, since the activity of the KAI1 fragment is determined by the Y*C motif and the EED motif, the KAI1 fragment may be in various forms as long as it comprises the two motifs. Therefore, the KAI1 fragment may be a peptide having the following structural formula:

| | [Structural formula 1] |
|---|---|
| (SEQ ID NO: 43) | |
| | Tyr Xaa1 Cys Xaa2 Xaa3 Xaa4 Xaa5 Xaa6 Xaa7 Glu |
| | Glu Asp Xaa1 may be Ala, Asp, Glu, Gly, Phe, |
| | Leu, Ser, Tyr, Cys, Trp, Pro, His, Gln, Arg, |
| | Ile, Met, Asn, Lys, Ser or Val. Preferably, |
| | Xaa1 may be Pro. |
| | Xaa2 may be Ala, Asp, Glu, Gly, Phe, Leu, Ser, |
| | Tyr, Cys, Trp, Pro, His, Gln, Arg, Ile, Met, |
| | Asn, Lys, Ser or Val. Preferably, Xaa2 may be |
| | Ser. |
| | Xaa3 may be Ala, Asp, Glu, Gly, Phe, Leu, Ser, |
| | Tyr, Cys, Trp, Pro, His, Gln, Arg, Ile, Met, |
| | Asn, Lys, Ser or Val. Preferably, Xaa3 may be |
| | Cys. |
| | Xaa4 may be Ala, Asp, Glu, Gly, Phe, Leu, Ser, |
| | Tyr, Cys, Trp, Pro, His, Gln, Arg, Ile, Met, |
| | Asn, Lys, Ser or Val. Preferably, Xaa4 may be |
| | Glu. |
| | Xaa5 may be Ala, Asp, Glu, Gly, Phe, Leu, Ser, |
| | Tyr, Cys, Trp, Pro, His, Gln, Arg, Ile, Met, |
| | Asn, Lys, Ser or Val. Preferably, Xaa5 may be |
| | Val. |
| | Xaa6 may be Ala, Asp, Glu, Gly, Phe, Leu, Ser, |
| | Tyr, Cys, Trp, Pro, His, Gln, Arg, Ile, Met, |
| | Asn, Lys, Ser or Val. Preferably, Xaa6 may be |
| | Lys. |
| | Xaa7 may be Ala, Asp, Glu, Gly, Phe, Leu, Ser, |
| | Tyr, Cys, Trp, Pro, His, Gln, Arg, Ile, Met, |
| | Asn, Lys, Ser or Val. Preferably, Xaa7 may be |
| | Gly. |

Another aspect of the present invention may be a pharmaceutical composition for preventing or treating hepatic fibrosis, comprising a polynucleotide encoding the KAI1 protein or a fragment thereof.

As used herein, the term “hepatic fibrosis (liver fibrosis)” refers to a symptom in which fibrous tissue proliferates due to damage to the liver. Hepatic fibrosis may progress to hepatic cirrhosis.

The hepatic fibrosis may be hepatic cirrhosis (liver cirrhosis). The hepatic cirrhosis refers to a disease in which the soft liver changes into a hard, rock-like liver that does not properly perform its original function of the liver. The hepatic cirrhosis may be selected from the group consisting of viral hepatic fibrosis, alcoholic hepatic cirrhosis, non-alcoholic hepatic cirrhosis, Wilson's disease-related hepatic cirrhosis, hemochromatosis-related hepatic cirrhosis, portal hepatic cirrhosis, post-necrosis hepatic cirrhosis, nutritional deficiency-related hepatic cirrhosis, and cardiac hepatic cirrhosis.

As used herein, the term “prevention” refers to any action that inhibits the occurrence of hepatic fibrosis or delays its onset by administering the pharmaceutical composition. The term “treatment” refers to any action that improves or beneficially changes the symptoms of hepatic fibrosis by administering the pharmaceutical composition.

The pharmaceutical composition may comprise an effective amount of KAI1 protein or a fragment thereof. The term “effective amount” refers to an amount sufficient to produce a prophylactic or therapeutic effect when administered to a subject in need of prevention or treatment. The effective amount can be appropriately selected by those of ordinary skill in the art depending on the cell or subject to be selected. The effective amount may be determined based on factors including the severity of the disease, the age, body weight, health, and gender of the patient, the patient's sensitivity to the drug, the administration time, the route of administration and excretion rate, the treatment period, composition used and drugs combined or used simultaneously, and other factors well known in the medical field. The effective amount may be about 0.5 μg to about 2 g, about 1 μg to about 1 g, about 10 μg to about 500 mg, about 100 μg to about 100 mg, or about 1 mg to about 50 mg per the pharmaceutical composition.

The pharmaceutical composition may further comprise known active ingredients having anti-inflammatory or antiviral activity.

In addition, the pharmaceutical composition may further comprise a pharmaceutically acceptable salt or carrier.

As used herein, the term “salt” refers to an addition salt of an inorganic acid salt, organic acid salt, or metal salt of a compound. The salt may be a pharmaceutically acceptable salt. The pharmaceutically acceptable salt may be a salt that does not cause serious irritation to the organism to which the compound is administered and does not impair the biological activity and physical properties of the compound. The inorganic acid salt may be hydrochloride, bromate, phosphate, sulfate, or disulfate. The organic acid salts may be formate, acetate, propionate, lactate, oxalate, tartrate, malate, maleate, citrate, fumarate, besylate, camsylate, edicylate, trichloroacetate, trifluoroacetate, benzoate, gluconate, methanesulfonate, glycolate, succinate, 4-toluenesulfonate, galacturonate, embonate, glutamate, methanesulfonic acid, ethanesulfonic acid, benzenesulfonic acid, p-toluenesulfonic acid, or aspartic acid salt. The metal salt may be a calcium salt, sodium salt, magnesium salt, strontium salt, or potassium salt.

The carrier is used to include an excipient, a diluent, or an auxiliary agent. For example, the carrier may be selected from the group consisting of lactose, dextrose, sucrose, sorbitol, mannitol, xylitol, erythritol, maltitol, starch, gum acacia, alginate, gelatin, calcium phosphate, calcium silicate, cellulose, methyl cellulose, polyvinylpyrrolidone, water, physiological saline, buffer such as PBS, methyl hydroxybenzoate, propyl hydroxybenzoate, talc, magnesium stearate, and mineral oil. The preparation may include a filling agent, an anti-coagulant, a lubricant, a wetting agent, a flavoring agent, an emulsifier, a preservative, or a combination thereof.

Meanwhile, the pharmaceutical composition of the present invention may be administered in a therapeutically effective amount.

As used herein, the term “administration” means introducing a predetermined substance into a subject by an appropriate method, and the composition may be administered through any general route as long as it can reach the target tissue. The pharmaceutical composition may be administered to a subject in a conventional manner via oral, transdermal, subcutaneous, rectal, intravenous, intraarterial, intraperitoneal, intramuscular, intrasternal, topical, or intradermal routes, but is not limited thereto. In addition, the pharmaceutical composition may be administered once per day, 2 to 24 times per day, 1 to 2 times every 3 days, 1 to 6 times per week, 1 to 10 times every 2 weeks, 1 to 15 times every 3 weeks, 1 to 3 times every 4 weeks, or 1 to 12 times per year. For example, the dosage of the pharmaceutical composition may be within the range of about 0.001 mg/kg to about 100 mg/kg, about 0.01 mg/kg to about 10 mg/kg, or about 0.1 mg/kg to about 1 mg/kg based on adults. The administration may be performed once per day, 2 to 24 times per day, 1 to 2 times every 3 days, 1 to 6 times per week, 1 to 10 times every 2 weeks, 1 to 15 times every 3 weeks, 1 to 3 times every 4 weeks, or 1 to 12 times per year. The dosage should not be construed as limiting the scope of the present invention in any respect.

The subject may be a subject who has hepatic fibrosis or is at risk of developing hepatic fibrosis. The subject may be a mammal, such as a human, cow, horse, pig, dog, sheep, goat or cat. Preferably, it may be a human.

The pharmaceutical composition may be prepared in any formulation according to conventional methods. The pharmaceutical composition may be formulated, for example, as a parenteral formulation (for example, an injection). In addition, the formulation may be prepared as a systemic formulation or a topical formulation. The pharmaceutical composition may be an injection for subcutaneous administration, intramuscular administration, or intravenous administration. The pharmaceutical composition may be a hard capsule, a soft capsule, a gel, a liquid formulation, or a spray formulation.

In another aspect of the present invention, there is provided the use of KAI1 protein or a fragment thereof for the prevention or treatment of hepatic fibrosis. In this case, the hepatic fibrosis, the prevention, the treatment, the KAI1 protein, and the fragment of the KAI1 protein are as described above.

In another aspect of the present invention, there is provided a method for preventing or treating hepatic fibrosis, comprising administering to a subject a pharmaceutical composition comprising KAI1 protein or a fragment thereof. In this case, the KAI1 protein, the fragment of the KAI1 protein, the pharmaceutical composition, the hepatic fibrosis, the prevention, the treatment, and the administration are as described above.

The subject may be a subject who has hepatic fibrosis or is at risk of developing hepatic fibrosis. The subject may be a mammal, such as a human, cow, horse, pig, dog, sheep, goat or cat. Preferably, it may be a human.

The protein or a fragment thereof may be administered to a subject in various ways and amounts depending on the route of administration, the dosage, and the frequency of administration, the patient's condition, and the presence or absence of side effects, and The optimal administration method, dosage and frequency of administration can be selected within an appropriate range by those of ordinary skill in the art. In addition, the pharmaceutical composition may be administered in combination with any known compound or natural extract useful for treating or preventing hepatic fibrosis.

In another aspect of the present invention, there is provided a KAI1 protein fragment of structural formula 1 above.

### Mode for Carrying out the Invention

Hereinafter, the present invention will be described in more detail by way of the following examples. However, the following examples are only for illustrating the present invention, and the scope of the present invention is not limited thereto.

### Example 1

### Design of KAI1 Peptide With Angiogenesis Inhibitory Function and Search of Functional Region Sequence

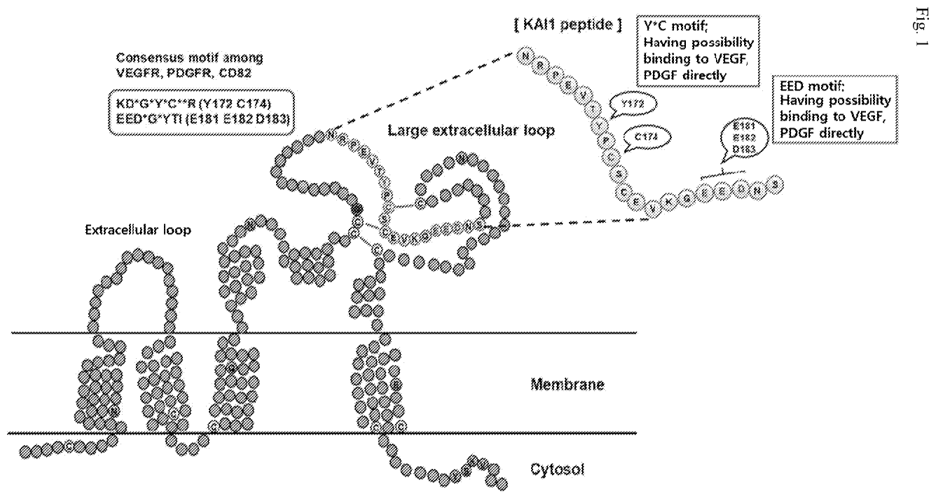

The large extracellular loop of KAI1 (SEQ ID NO: 1); and the conserved motif (consensus motif) between PDGFR and VEGFR were identified by homology analysis. It was confirmed by homology analysis that among the conserved motifs in the KAI1 protein, the Y*C motif and EED motif are sites that can directly bind to VEGF (vascular endothelial growth factor) or PDGF (platelet-derived growth factor) (FIG. 1).

A functional KAI1 peptide (wild type peptide, pepKAI1 WT) consisting of 20 amino acids comprising the Y*C motif and EED motif, and a mutant peptide (pepKAI1 Mut) in which both Y*C and EED were substituted with alanine (A) were constructed. Information on the peptide sequence is shown in Table 1 below. In Table 1, amino acid sequences that differ between the wild type KAI1 peptide sequence and the mutant KAI1 peptide sequence are underlined.

TABLE 1

| | Amino acid sequence |
|---|---|
| Peptide | (N terminus -> C terminus) |
| wild type KAI1 | NRPEVTYPCSCEVKGEEDNS |
| peptide sequence | (SEQ ID NO: 2) |
| mutant KAI1 | NRPEVTAPASCEVKGAAANS |
| peptide sequence | (SEQ ID NO: 3) |

### Example 2

### Confirmation of Activity of KAI1 Protein to Inhibit Hepatic Fibrosis in Acute Hepatic Fibrosis Animal Model

### Example 2.1

### Acute Hepatic Fibrosis Animal Model Experiment

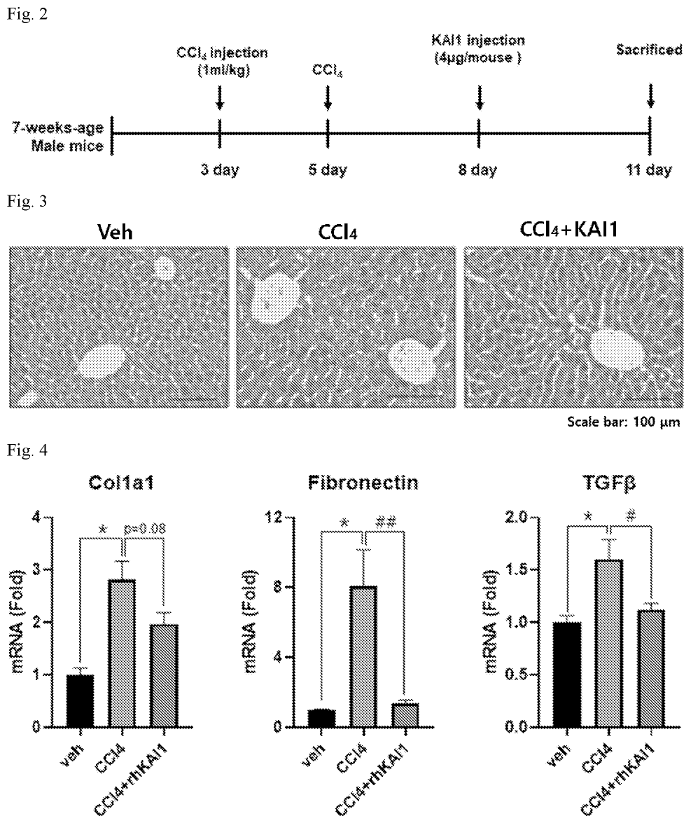

In order to verify the effect of inhibiting hepatic fibrosis by KAI1 protein, an animal experiment was conducted as follows (FIG. 2).

Specifically, 7-week-old C57BL/6 male mice was prepared, and CCl₄ was diluted in corn oil at a concentration of 1 mL/kg. In order to induce acute liver injury, the mice were injected intraperitoneally with the diluted CCl₄ twice a week. As a control group, the mice were intraperitoneally injected with the same amount of the corn oil.

As a KAI1 protein, a polypeptide (Human CD82/KAI-1 Protein (His Tag), Sino Biological Inc.) comprising a KAI1 fragment comprising a polypeptide (extracellular domain, SEQ ID NO: 4) consisting of the 111st to 228th amino acid sequence from the N terminus in a polypeptide consisting of the amino acid sequence of SEQ ID NO: 1 was used. On the 8th day after the start of the experiment, 4 μg of KAI1 protein was injected intraperitoneally per mouse, and on the 11th day, the mice were sacrificed and liver tissue was extracted.

### Example 2.2

### Inhibition of Fibrosis by Treatment With KAI1 Protein in Acute Hepatic Fibrosis Model

The liver tissue extracted in Example 2.1. was fixed in 10% (v/v) formalin solution. Thereafter, the liver tissue was embedded in paraffin and sectioned to a thickness of 4 μm to prepare tissue sections. After deparaffinization and superhydration, H&E (hematoxylin & eosin) staining was performed and observed under an optical microscope.

As a result, as shown in FIG. 3, the degree of cell damage and inflammatory cell infiltration was increased in the control group (Veh), but the degree was reduced in the group treated with KAI1 protein (CCl₄+KAI1). Through the above results, it was confirmed that acute hepatic fibrosis is inhibited by the KAI1 protein.

### Example 2.3

### Inhibition of Fibrosis by Treatment With KAI1 Protein in Acute Hepatic Fibrosis Model

In order to confirm whether KAI1 may inhibit fibrosis increased by CCl₄ in liver tissue, differences in expression of fibrosis-related genes were confirmed.

RNA was obtained from the liver tissue extracted in Example 2.1. The RNA from the liver tissue was obtained using Favorgen® Trizol according to the manufacturer's instructions. The RNA was reverse transcribed into cDNA using a cDNA synthesis kit (Bioneer corp.). Thereafter, real-time PCR (qPCR) was performed to confirm changes in the expression levels of fibrosis-related genes Col1a1 (collagen type 1), fibronectin, and TGFβ1 (transforming growth factor-β1). At this time, information on the primers used is shown in Table 2 below.

TABLE 2

| | Primer | |
|---|---|---|
| Item | Sequence | |
| mouse | forward | 5′-TGATACGCCTGAGTGGCTGTCT-3′ |
| TGFβ1 | | (SEQ ID NO: 5) |
| | reverse | 5′-CACAAGAGCAGTGAGCGCTGAA-3′ |
| | | (SEQ ID NO: 6) |
| mouse | forward | 5′-CCCTATCTCTGATACCGTTGTCC-3′ |
| fibronectin | | (SEQ ID NO: 7) |
| | reverse | 5′-TGCCGCAACTACTGTGATTCGG-3′ |
| | | (SEQ ID NO: 8) |
| mouse | forward | 5′-CCTCAGGGTATTGCTGGACAAC-3′ |
| Colla1 | | (SEQ ID NO: 9) |
| | reverse | 5′-CAGAAGGACCTTGTTTGCCAGG-3′ |
| | | (SEQ ID NO: 10) |
| mouse | forward | 5′-CATCACTGCCACCCAGAAGACTG-3′ |
| GAPDH | | (SEQ ID NO: 11) |
| | reverse | 5′-ATGCCAGTGAGCTTCCCGTTCAG-3′ |
| | | (SEQ ID NO: 12) |

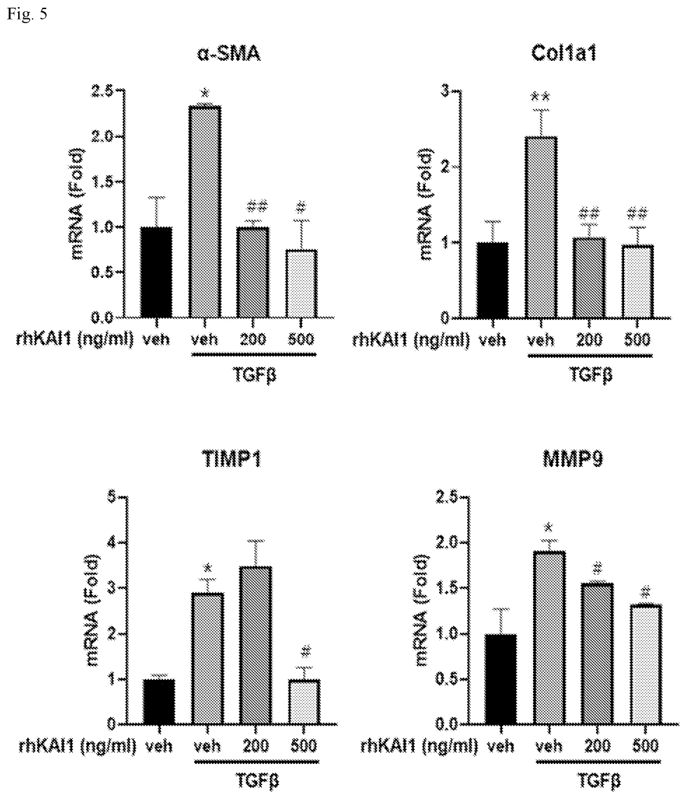

As a result, as shown in FIG. 4, it was confirmed that the gene expression of TGF-β1, fibronectin, and Col1a1 was increased by treatment with CCl₄, while the gene expression was reduced by treatment with KAI1. Through the above results, it was confirmed that the KAI1 protein may inhibit hepatic fibrosis.

### Example 3

### Confirmation of Activity of KAI1 Protein to Inhibit Hepatic Fibrosis

### Example 3.1

### Inhibition of Fibrosis-Related Gene Expression by Treatment With KAI1 Protein in Hepatic Fibrosis Cell Model

In order to confirm the activity of KAI1protein to inhibit hepatic fibrosis at the cellular level, the liver cell line (AML12), which had hepatic fibrosis induced by treatment with TGF-β1, was treated with KAI1 protein to confirm changes in the expression of fibrosis-related genes.

Specifically, the AML 12 cells were cultured in DMEM/F12 medium containing 10% (v/v) fetal bovine serum (FBS), 1% (w/v) penicillin-streptomycin, 10 μg/mL insulin, 5.5 μg/mL transferrin, 5 ng/mL selenium, and 40 ng/ml dexamethasone. The AML12 cells were treated with KAI1 protein at a concentration of 200 ng/ml or 500 ng/mL, respectively, and reacted for 2 hours. Thereafter, the cells were treated with TGF-β1, a fibrosis-inducing cytokine, at a concentration of 2 ng/mL and cultured for an additional 24 hours, and then the cells were collected. RNA from the collected cells was extracted with Trizol, cDNA was synthesized, and then changes in expression of fibrosis-related genes (α-SMA, Col1a1, TIMP1, and MMP9) were confirmed by qPCR analysis. At this time, information on the primers used in qPCR is shown in Table 3 below.

TABLE 3

| | Primer | |
|---|---|---|
| Item | Sequence | |
| mouse | forward | 5′-TGCTGACAGAGGCACCACTGAA-3′ |
| α-SMA | | (SEQ ID NO: 13) |
| | reverse | 5′-CAGTTGTACGTCCAGAGGCATAG-3′ |
| | | (SEQ ID NO: 14) |
| mouse | forward | 5′-CCTCAGGGTATTGCTGGACAAC-3′ |
| Colla1 | | (SEQ ID NO: 9) |
| | reverse | 5′-CAGAAGGACCTTGTTTGCCAGG-3′ |
| | | (SEQ ID NO: 10) |
| mouse | forward | 5′-GATATGCCCACAAGTCCCAGAACC-3′ |
| TIMP1 | | (SEQ ID NO: 15) |
| | reverse | 5′-GCACACCCCACAGCCAGCACTAT-3′ |
| | | (SEQ ID NO: 16) |
| mouse | forward | 5′-GCTGACTACGATAAGGACGGCA-3′ |
| MMP9 | | (SEQ ID NO: 17) |
| | reverse | 5′-TAGTGGTGCAGGCAGAGTAGGA-3′ |
| | | (SEQ ID NO: 18) |
| mouse | forward | 5′-CATCACTGCCACCCAGAAGACTG-3′ |
| GAPDH | | (SEQ ID NO: 11) |
| | reverse | 5′-ATGCCAGTGAGCTTCCCGTTCAG-3′ |
| | | (SEQ ID NO: 12) |

As a result, as shown in FIG. 5, the expression of fibrosis-related genes, which had been increased by treatment with TGF-β1, was reduced by treatment with KAI1 protein. Through the above results, it was confirmed that the KAI1 protein mayn inhibit fibrosis at the liver cell level.

### Example 3.2

### Inhibition of Epithelial-Mesenchymal Transition-Related Protein Expression by Treatment With KAI1 Protein in Hepatic Fibrosis Cell Model

The epithelial-mesenchymal transition (EMT) process has been proposed as one of the mechanisms of fibrosis. Therefore, the effect of KAI1 protein on the EMT process induced by TGF-1 in mouse liver cell line (AML12) was confirmed.

Specifically, the AML12 cells were treated with of KAI1 protein at a concentration of 500 ng/ml and reacted for 2 hours. Thereafter, the cells were treated with TGF-β1 and cultured for an additional 24 hours or 48 hours, and then the cells were collected. The collected cells were lysed in RIPA buffer containing protease inhibitors to extract proteins, and immunoblotting was performed by loading an equal amount of proteins. The protein expression level of ZO-1 was confirmed using an antibody of ZO-1 (anti-ZO antibody, 13663, Abcam), an epithelial biomarker during the EMT process. At this time, anti-tubulin antibody (T5168, Sigma-Aldrich) was used as an internal control, and HRP-conjugated anti-rabbit antibody (ADI-SAB-300-J, Enzo Life Sciences) was used as a secondary antibody.

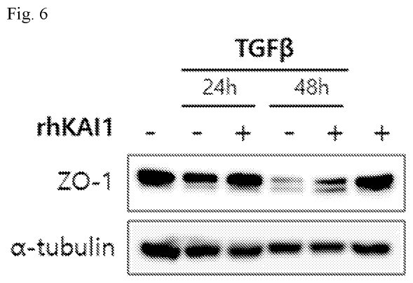

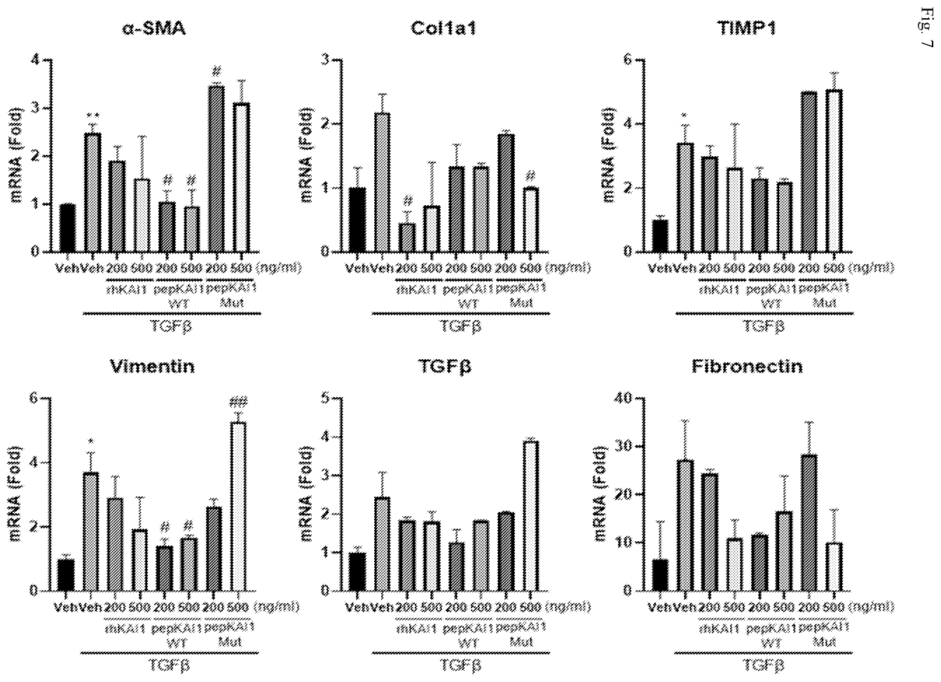

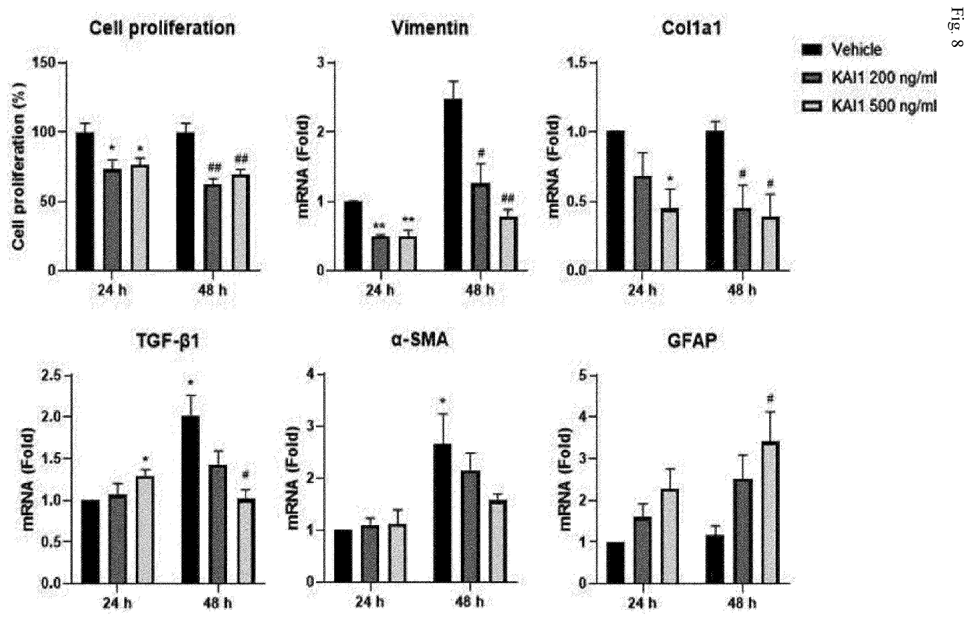

As a result, as shown in FIG. 6, the expression of ZO-1 protein, which had been reduced by treatment with TGF-β1, was recovered by treatment with KAI1 protein. Through the above results, it was confirmed that KAI1 may inhibit fibrosis in liver cells by inhibiting the EMT process.

### Example 3.3

Inhibition of Fibrosis Gene Expression by Treatment with KAI1 Protein in Hepatic Fibrosis Cell Model

The effect of wild type KAI1 peptide (pepKAI1 WT) or mutant KAI1 peptide (pepKAI1 Mut) on fibrosis induced by treatment with TGF-β1 was confirmed in mouse liver cell line (AML12).

Specifically, the AML 12 cells were treated with recombinant human KAI1 protein (rhKAI1), wild type KAI1 peptide (pepKAI1 WT), and mutant KAI1 peptide (pepKAI1 Mut) at a concentration of 200 ng/mL or 500 ng/mL, respectively, and reacted for 2 hours. Thereafter, the cells were treated with TGF-β1 at a concentration of 2 ng/ml and cultured for an additional 24 hours, and then the cells were collected. RNA from the collected cells was extracted with Trizol, cDNA was synthesized, and then changes in expression of fibrosis-related genes (α-SMA, Col1a1, TIMP1, Vimentin, TGF-β, and fibronectin) were confirmed by qPCR analysis. At this time, information on the primers used in qPCR is shown in Table 4 below.

TABLE 4

| | Primer | |
|---|---|---|
| Item | Sequence | |
| mouse | forward | 5′-TGCTGACAGAGGCACCACTGAA-3′ |
| α-SMA | | (SEQ ID NO: 13) |
| | reverse | 5′-CAGTTGTACGTCCAGAGGCATAG-3′ |
| | | (SEQ ID NO: 14) |
| mouse | forward | 5′-CCTCAGGGTATTGCTGGACAAC-3′ |
| Colla1 | | (SEQ ID NO: 9) |
| | reverse | 5′-CAGAAGGACCTTGTTTGCCAGG-3′ |
| | | (SEQ ID NO: 10) |
| mouse | forward | 5′-GATATGCCCACAAGTCCCAGAACC-3′ |
| TIMP1 | | (SEQ ID NO: 15) |
| | reverse | 5′-GCACACCCCACAGCCAGCACTAT-3′ |
| | | (SEQ ID NO: 16) |
| mouse | forward | 5′-CGGAAAGTGGAATCCTTGCAGG-3′ |
| Vimentin | | (SEQ ID NO: 19) |
| | reverse | 5′-AGCAGTGAGGTCAGGCTTGGAA-3′ |
| | | (SEQ ID NO: 20) |
| mouse | forward | 5′-TGATACGCCTGAGTGGCTGTCT-3′ |
| TGFβ | | (SEQ ID NO: 5 |
| | reverse | 5′-CACAAGAGCAGTGAGCGCTGAA-3′ |
| | | (SEQ ID NO: 6) |
| mouse | forward | 5′-CCCTATCTCTGATACCGTTGTCC-3′ |
| fibronectin | | (SEQ ID NO: 7) |
| | reverse | 5′-TGCCGCAACTACTGTGATTCGG-3′ |
| | | (SEQ ID NO: 8) |
| mouse | forward | 5′-CATCACTGCCACCCAGAAGACTG-3′ |
| GAPDH | | (SEQ ID NO: 11) |
| | reverse | 5′-ATGCCAGTGAGCTTCCCGTTCAG-3′ |
| | | (SEQ ID NO: 12) |

As a result, as shown in FIG. 7, the expression of fibrosis-related genes, which had been increased by treatment with TGF-β1, was reduced by treatment with wild type KAI1 peptide (pepKAI1 WT). The gene expression in the group treated with wild type KAI1 peptide (pepKAI1 WT) was further reduced compared to the group treated with rhKAI1 protein. On the other hand, it was confirmed that gene expression in the group treated with mutant KAI1 peptide (pepKAI1 Mut) was not reduced.

Through the above results, it was confirmed that KAI1 protein may be used as a therapeutic agent for inhibiting hepatic fibrosis.

### Example 4

### Confirmation of Effect of Inhibiting Fibrosis by Treatment With KAI1 Protein

### Example 4.1

### Confirmation of Effect of Inhibiting Cell Proliferation and Fibrosis-Related Gene Expression by Treatment With KAI1 Protein in Hepatic Stellate Cell Line

It is known that fibrosis occurs due to excessive proliferation and activation of hepatic stellate cells. Therefore, changes in fibrosis-related gene expression by treatment with KAI1 protein were confirmed in human hepatic stellate cell line (LX2).

Specifically, the LX2 cell line was treated with recombinant human KAI1 protein (rhKAI1) at a concentration of 200 ng/mL or 500 ng/mL, respectively. Thereafter, the cells were cultured for 24 hours or 48 hours and collected to confirm cell proliferation and fibrosis-related gene expression in the cells. The cell proliferation was measured using MTS assay. RNA from the collected cells was extracted with Trizol, cDNA was synthesized, and then changes in expression of fibrosis-related genes (α-SMA, Col1a1, Vimentin, TGF-β, and GFAP) were confirmed by qPCR analysis. At this time, information on the primers used in qPCR is shown in Table 5 below.

TABLE 5

| | Primer | |
|---|---|---|
| Item | Sequence | |
| human | forward | 5′-TTGTGGCAGGGTTGATGTTA-3′ |
| Vimentin | | (SEQ ID NO: 21) |
| | reverse | 5′-CACCCACTCCAACTCCAACT-3′ |
| | | (SEQ ID NO: 22) |
| human | forward | 5′-TTATAGAGCGATACAAGGGGGAG-3′ |
| Colla1 | | (SEQ ID NO: 23) |
| | reverse | 5′-CGCCGTCTGATTATCTTGATGAG-3′ |
| | | (SEQ ID NO: 24) |
| human | forward | 5′-GAGGCTACCCTAGACACAAGG-3′ |
| TGFβ | | (SEQ ID NO: 25) |
| | reverse | 5′-GGGTGCCGTTGCTCATCATA-3′ |
| | | (SEQ ID NO: 26) |
| human | forward | 5′-TGAACTCCAACGTCAAGCGG-3′ |
| α-SMA | | (SEQ ID NO: 27) |
| | reverse | 5′-CGCCGTCTGATTATCTTGATGAG-3′ |
| | | (SEQ ID NO: 28) |
| human | forward | 5′-CTGGAGGTTGAGAGGGACAA-3′ |
| GFAP | | (SEQ ID NO: 29) |
| | reverse | 5′-CAGCCTCAGGTGGTTTCAT-3′ |
| | | (SEQ ID NO: 30) |
| human | forward | 5′-GACTCCGGAACAAACGTGAGGT-3′ |
| 9S | | (SEQ ID NO: 31) |
| | reverse | 5′-CTTCATCTTGCCCTCGTCCA-3′ |
| | | (SEQ ID NO: 32) |

As a result, as shown in FIG. 8, cell proliferation was significantly reduced by treatment with KAI1 protein. In addition, the expression of fibrosis-related genes Vimentin, Col1a1, TGF-β, and α-SMA was also significantly reduced. On the other hand, the expression of GFAP, a marker gene for resting stellate cells, showed a tendency to increase.

### Example 4.2

Confirmation of Effect of Inhibiting Fibrosis-Related Protein Expression by Treatment with KAI1 Protein in Hepatic Stellate Cell Line

The expression of fibrosis-related proteins was confirmed in human hepatic stellate cell line (LX2) treated with KAI1 protein in the same manner as in Example 4.1. At this time, anti-Vimentin antibody (sc-5565, Santa Cruz Biotech.), anti-Col1α1 antibody (NBP1-30054, Novus Biological), anti-α-SMA antibody (A5228, Sigma-Aldrich), anti-GFAP antibody (Z0334, Dako), and anti-GAPDH antibody (MCA4739, AbD Serptec) were used as primary antibodies, and HRP-conjugated anti-rabbit antibody (ADI-SAB-300-J, Enzo Life Sciences) and anti-mouse antibody (ADI-SAB-100-J) were used as secondary antibodies.

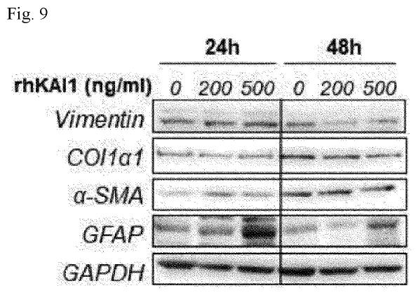

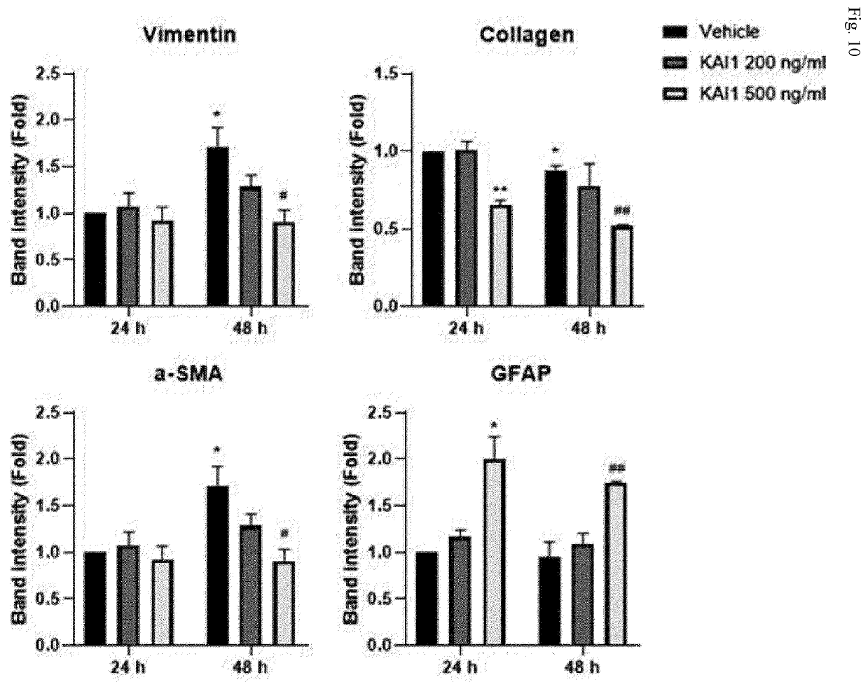

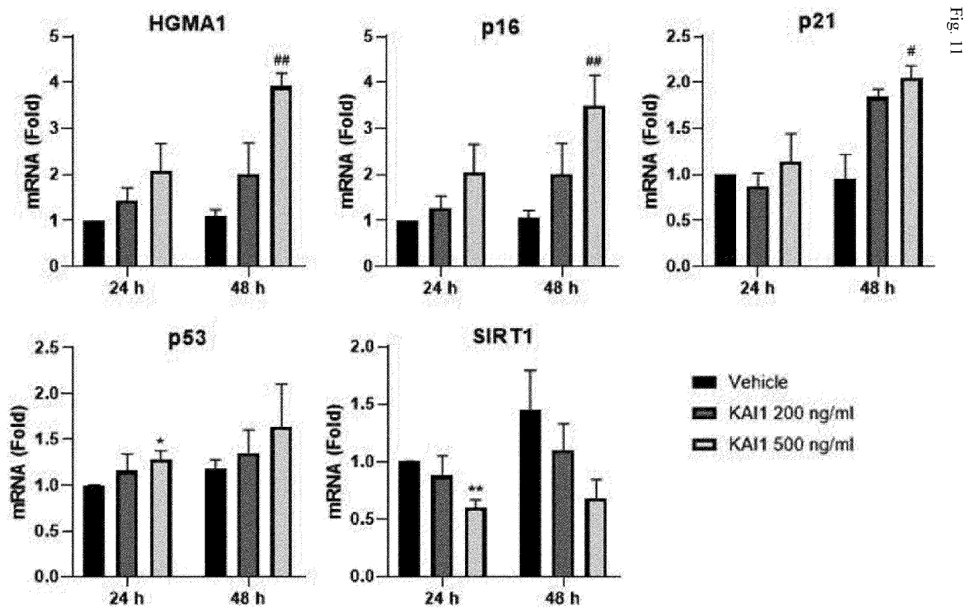

As a result, as shown in FIGS. 9 and 10, it was confirmed that the expression of fibrosis-related proteins (Vimentin, Col1α1, and α-SMA) was reduced in hepatic stellate cell lines by treatment with KAI1 protein compared to the untreated group, while the expression of GFAP, a marker gene for resting hepatic stellate cells, was increased. The above results are consistent with the results for the expression of fibrosis-related proteins in Example 4.1.

### Example 4.3

### Confirmation of Effect Of Inhibiting Senescence-Related Gene Expression by Treatment With KAI1 Protein in Hepatic Stellate Cell Line

It is known that hepatic stellate cells can inhibit hepatic fibrosis by inducing senescence in the liver. Therefore, the expression of senescence-related genes (HGMA1, p16, p21 and p53, SIRT1) was confirmed in human hepatic stellate cell line (LX2) treated with KAI1 protein in the same manner as in Example 4.1. At this time, information on the primers used in qPCR is shown in Table 6 below.

TABLE 6

| | Primer | |
|---|---|---|
| Item | Sequence | |
| human | forward | 5′-CAACTCCAGGAAGGAAACCA-3′ |
| HGMA1 | | (SEQ ID NO: 33) |
| | reverse | 5′-AGGACTCCTGCGAGATGC-3′ |
| | | (SEQ ID NO: 34) |
| human | forward | 5′-CACCGAATAGTTACGGTCGG-3′ |
| p16 | | (SEQ ID NO: 35) |
| | reverse | 5′-GCACGGGTCGGGTGAGAGTG-3′ |
| | | (SEQ ID NO: 36) |
| human | forward | 5′-CATGTGGACCTGTCACTGTCTTGTA-3′ |
| p21 | | (SEQ ID NO: 37) |
| | reverse | 5′-GAAGATCAGCCGGCGTTTG-3′ |
| | | (SEQ ID NO: 38) |
| human | forward | 5′-CCTCAGCATCTTATCCGAGTGG-3′ |
| p53 | | (SEQ ID NO: 39) |
| | reverse | 5′-TGGATGGTGGTACAGTCAGFGC-3′ |
| | | (SEQ ID NO: 40) |
| human | forward | 5′-TGCTGGCCTAATAGAGTGGCA-3′ |
| SIRT1 | | (SEQ ID NO: 41) |
| | reverse | 5′-CTCAGCGCCATGGAAAATGT-3′ |
| | | (SEQ ID NO: 42) |
| human | forward | 5′-GACTCCGGAACAAACGTGAGGT-3′ |
| 9S | | (SEQ ID NO: 31) |
| | reverse | 5′-CTTCATCTTGCCCTCGTCCA-3′ |
| | | (SEQ ID NO: 32) |

As a result, as shown in FIG. 11, it was confirmed that the expression of senescence marker genes HGMA1, p16, p21, and p53 was increased in the group treated with KAI1 protein compared to the control group, while the expression of SIRT1, a senescence inhibition marker, was reduced.

### Example 4.4

### Confirmation of Effect of Inhibiting Senescence-Related Protein Expression by Treatment With KAI1 Protein in Hepatic Stellate Cell Line

The expression of senescence-related proteins was confirmed in human hepatic stellate cell line (LX2) treated with KAI1 protein in the same manner as in Example 4.1. At this time, anti-SIRT1 antibody (ab32441, Abcam), anti-p16 antibody (10883-1-ap, Protein tech.), and anti-GAPDH antibody (MCA4739, AbD Serptec) were used as primary antibodies, and HRP-conjugated anti-rabbit antibody (ADI-SAB-300-J, Enzo Life Sciences) and anti-mouse antibody (ADI-SAB-100-J) were used as secondary antibodies.

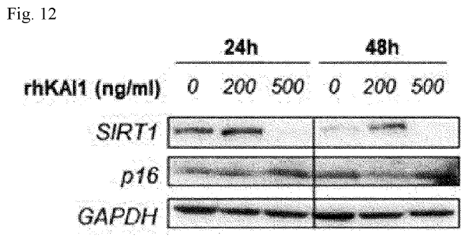

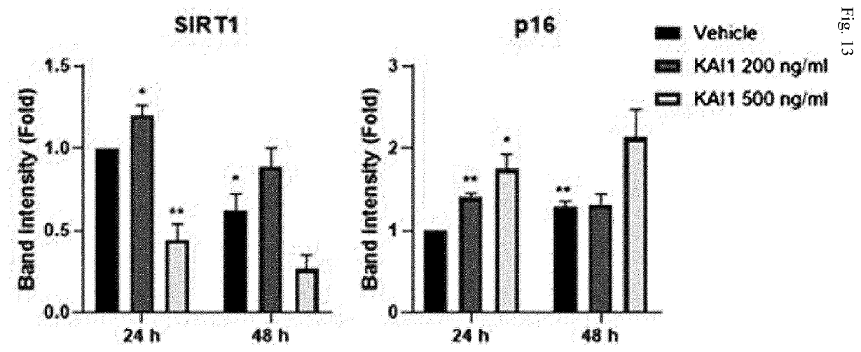

As a result, as shown in FIGS. 12 and 13, it was confirmed that the expression of p16, a senescence marker protein, was increased in the group treated with KAI1 protein compared to the untreated group, while the expression of SIRT1, a senescence inhibition marker protein, was reduced. The above results are consistent with the results for the expression of senescence-related genes in Example 4.3.

Through the above results, it was confirmed that KAI1 protein may inhibit hepatic fibrosis by inhibiting the activity of hepatic stellate cells, which are closely related to the hepatic fibrosis process, and inducing senescence.

1. A pharmaceutical composition for preventing or treating hepatic fibrosis, comprising KAI1 protein or a fragment thereof.

2. The pharmaceutical composition for preventing or treating hepatic fibrosis according to claim 1, wherein the KAI1 protein comprises a polypeptide consisting of the amino acid sequence of SEQ ID NO: 1.

3. The pharmaceutical composition for preventing or treating hepatic fibrosis according to claim 1, wherein the fragment of the KAI1 protein comprises a polypeptide consisting of the amino acid sequence of SEQ ID NO: 2 or SEQ ID NO: 4.

4. The pharmaceutical composition for preventing or treating hepatic fibrosis according to claim 1, wherein the hepatic fibrosis is hepatic cirrhosis.

5. The pharmaceutical composition for preventing or treating hepatic fibrosis according to claim 4, wherein the hepatic cirrhosis is selected from the group consisting of viral hepatic fibrosis, alcoholic hepatic cirrhosis, non-alcoholic hepatic cirrhosis, Wilson's disease-related hepatic cirrhosis, hemochromatosis-related hepatic cirrhosis, portal hepatic cirrhosis, post-necrosis hepatic cirrhosis, nutritional deficiency-related hepatic cirrhosis, and cardiac hepatic cirrhosis.

6. Use of KAI1 protein or a fragment thereof for the prevention or treatment of hepatic fibrosis.

7. A method for preventing or treating hepatic fibrosis, comprising administering to a subject a pharmaceutical composition comprising KAI1 protein or a fragment thereof.

8. The method of claim 7, wherein the KAI1 protein comprises a polypeptide consisting of the amino acid sequence of SEQ ID NO: 1.

9. The method of claim 7, wherein the fragment of the KAI1 protein comprises a polypeptide consisting of the amino acid sequence of SEQ ID NO: 2 or SEQ ID NO: 4.

10. The method of claim 7, wherein the hepatic fibrosis is hepatic cirrhosis.

11. The method of claim 10, wherein the hepatic cirrhosis is selected from the group consisting of viral hepatic fibrosis, alcoholic hepatic cirrhosis, non-alcoholic hepatic cirrhosis, Wilson's disease-related hepatic cirrhosis, hemochromatosis-related hepatic cirrhosis, portal hepatic cirrhosis, post-necrosis hepatic cirrhosis, nutritional deficiency-related hepatic cirrhosis, and cardiac hepatic cirrhosis.
